(19) United States
(12) Patent Application Publication
(10) Pub. No.: US 2003/0093028 A1
(43) Pub. Date: May 15, 2003

(54) Appararus and method for magnetic induction of therapeutic electric fields

(76) Inventor: Michael Spiegel, Miami Beach, FL (US)

Correspondence Address: LERNER AND GREENBERG, P.A., POST OFFICE BOX 2480, HOLLYWOOD, FL, 33022-2480, US

(21) Appl. No.: 10/035,854

(22) Filed: Nov. 9, 2001

(51) Int. Cl. A61N 1/22; A61N 1/18

(52) U.S. Cl. 604/20; 607/3

(57) ABSTRACT

An apparatus and method utilize changing magnetic fields created either by arrays of spatially translating permanent magnets or by a series of sequentially energized electromagnets. The time changing magnetic fields induce electric currents throughout a treated volume of human blood, bone, tissue, organs, or nerves.

A first method utilizes permanent magnets mounted in a sequential stepwise manner and moved in space so that the movement and subsequent changing field strength of such magnets will induce an Electromotive force. The electromotive force will induce an electron current in the infused volume that is directly proportional to the strength of the magnetic field and the velocity of that field relative to the electrons at each point of the infused volume.

A second method utilizes a series of electromagnets fitted closely together and sequentially energized to effect a uniformly increasing induced magnetic field in the treatment volume.

### BACKGROUND OF THE INVENTION

1. Field of the Invention

This invention relates to applying electric fields to various parts of the body for medical treatment. More specifically, this invention applies a changing magnetic field on human tissue, wherein the time change of magnetic field strength (dB/dt) produces a resultant electromotive force (EMF) on the ions such as that caused by an electric field. Thus the invention treats human tissues with time changing magnetic fields that induce an electric field through known physical phenomena.

The invention also lies generally in the field of ion propulsion. More specifically, the invention relates to using a moving magnetic field such as those found in the area of plasma physics with certain applications concerned with rocket propulsion and controlled fusion. The application of this invention is not in these areas but is in a lower energy regime with application for the medicinal treatment of human bone, blood, tissue, and organs by the application of an electric field without the use of electrodes.

2. Description of the Prior Art

DC-like currents are recognized as providing medicinal benefits when applied to biological materials. For example, DC-like currents applied on or beneath the skin's surface have been effective in promoting rapid healing of bones, tissues, and even regrowth of spinal cord axons.

Typical apparatus have relied on electrode-imposed electric fields to impart the electromotive force required to produce the DC-like electric currents in treated tissues. The electrodes are inserted beneath the skin. For example, the use of electrodes to induce currents to treat spinal cord injuries is disclosed in Borgens et al., “Applied Electric Fields in Clinical Cases of Complete Paraplegia in Dogs.” Restorative Neurology and Neuroscience, Vol. 5, pp. 305-322 (1993).

The invasive nature of electrodes threatens the beneficial outcome of the clinical treatment and production of uniform electric fields within the treated tissue. For example, the electrodes can cause infection or become displaced. Moreover the electric fields produced are non-uniform in both intensity and geometry due to the polarization of the surrounding media at each electrode with oppositely charged ions that weaken and distort the electric field.

The basic concept for the beneficial application of a magnetically generated Lorentz force field to a volume is disclosed in Spiegel (U.S. Pat. No. 5,200,071). In contrast, the invention of the instant application discloses an invention that generates an equivalent electric field with both dynamic and time changing magnetic fields. The invention of the instant application further discloses an invention that generates an equivalent electric field that will stimulate the growth, repair, and renewal of damaged human bone, blood, tissue, and organs. The equivalent electric field also can be used to block specific nerve signals to eliminate pain. The equivalent electric field allows the transdermal transport of efficacious ionic components to specific locations within the tissue. The equivalent electric field can be increased to such power that it will destroy certain volumes of cancerous tissue.

Baermann et al. (U.S. Pat. No. 3,051,988) disclose a magnetic material manipulation device that is superficially similar in appearance to an embodiment of the device disclosed herein. Baermann's device is intended to handle magnetic material for industrial purposes. It uses low velocities and does not generate significant electrical fields. It has no specified medical application and has no mention or understanding of the field generation process disclosed herein.

In a series of patents, Ryaby et al. (U.S. Pat. No. 4,105,017; U.S. Pat. No. 4,266,532; U.S. Pat. No. 4,266,533; and U.S. Pat. No. 4,315,503) disclose a magnetically induced current of specific frequency and amplitude for clinically treating living tissue. The current is induced by an electromagnetic coil. As the coil is electrically energized, it generates a resultant time varying magnetic field. The electric field, and consequent electric current, induced by such a coil must be of a reversing nature. The system disclosed could never produce a DC or rectified AC electric current in treated tissue. Instead, Ryaby et al. produce pulses of induced AC current of specific frequencies and modulations that are purported to be clinically efficacious.

Ryaby et al. teach a medical treatment device that is capable of generating only AC current. The AC current is developed by a time varying single electromagnetic coil that is spatially static. It in no way suggests the development of electric currents through the use of spatially dynamic magnetic fields. It does not suggest the generation of electric fields with a stepwise changing magnetic field. It is the disclosure and unique application of such spatially dynamic and stepwise changing magnetic fields and their resultant asymmetric electric currents that distinguishes the current teaching over prior art.

Horl (U.S. Pat. No. 4,727,857) discloses a moving disk with a single set of opposite polarity permanent magnets on one or both faces of the disk. This device produces spatially dynamic magnetic fields that will generate a reversing sinusoidal electric field with each revolution of the disk.

It is the stated object of this teaching that it will produce “pulsating magnetic fields, which is capable of exerting radially inwardly or radially outwardly directed forces.” In contrast, Horl's teaching will produce only purely sinusoidal electric fields and currents near the surface of each magnetic pole face. It is clear from this teaching that it is neither understood nor intended that asymmetrical directed electric currents are an intended resultant clinical benefit. It is the sole intent of this teaching to provide such clinical benefit as may accrue through the action of a pulsing magnetic field of a singular geometry. Such electric fields as generated by this device will be sinusoidal and thus, as shown by cited studies, (Reich & Tarjan) of little therapeutic benefit.

In the article titled “Magnetic Field Therapy to Support Keratotomy,” Ivashina et al. teach to use a moving set of magnets to enhance recovery rates and reduce pain. The primary distinction between Ivashina and this invention is that the field generated by Ivashina is purely sinusoidal which can produce very limited charge transport. Ivashina et al. do not teach or suggest a “square wave” electric field that can produce significant charge transport of the type required by the DC-like current shown to be effective in the Reich & Tarjan study. The current disclosed invention is further distinguished by a continuous exposure to a magnetic array as opposed to the simple split fields of Ivashina et al. In addition, Ivashina et al. do not teach or suggest incorporating specifically designed permanent or electromagnets that generate dynamic and stepwise changing magnetic fields to induce a continuous and uniform DC-like “square wave” electric field.

All prior art, with the above noted exceptions, has attempted to treat the above clinical problems with the insertion of electrodes to generate an electric field. The breath and effectiveness of these methods are described in the following publications: “Electric Fields in Vertebrate Repair” And “The Body Electric”. Evidence of the potential application to regeneration of nerve tissue in mammals is most recently given by Borgens et al. in the report, “Applied electric fields . . . in Dogs.” All of these references agree that the most significant beneficial results are obtained through the application of DC currents that mimic the body's own mechanism.

### SUMMARY OF THE INVENTION

It is accordingly an object of the invention to provide an apparatus and methods for inducing DC-like electric fields in living cells, which overcomes the above-mentioned disadvantages of the heretofore-known devices and methods of this general type.

With the foregoing and other objects in view there is provided, in accordance with the invention, an apparatus that produces a DC-like electric field within biological material, such as living cells and tissue, to obtain a desired clinical benefit without the use of invasive electrodes. The invention allows for the generation of electric current without the use of invasive electrodes. This generated electric current mimics the natural mechanism of the cell of tissue. In addition, the present invention avoids the side effects associated with electrodes, such as infection and unwanted mineral deposits.

With the foregoing and other objects in view there is provided, in accordance with the invention, an apparatus for treating biological material. The apparatus includes two magnets and a drive. The magnets each produce a magnetic field that combines to form a magnetic field on the biological material. The drive is for changing the magnetic field on the biological material.

In accordance with a further object of the invention, a method for inducing a DC-like electric field in biological tissue includes the following steps. The first step is increasing a magnetic field on the biological tissue. The next step is suddenly decreasing the magnetic field. The method can then be cyclically repeated to create a magnetic field having a saw-tooth shaped intensity over time.

Generating beneficial and curative DC-like electric fields by moving and stepwise changing magnetic fields allows the treatment of patients without resorting to the dangerous invasive techniques required by direct application of equivalent electric fields.

Additional features and advantages of the invention will be set forth in the description which follows, and in part will be apparent from the description, and may be learned by the practice of the invention. The objects and advantages of the invention will be realized and attained by the device particularly pointed out in the written description and claims hereof as well as the appended drawings.

To achieve these and other advantages and in accordance with the purpose of the invention, as embodied and broadly described, the invention provides an apparatus of either permanent magnets or electromagnets arranged and prepared so as to cause the stepwise changing of arranged or generated magnetic fields relative to the tissue in which the desired DC-like electric filed is to be induced. The stepwise changing of such magnetic fields by the apparatus induces a force on electrons within living cells and tissue and generates a DC-like electric current in the cells or tissue.

In accordance with a further object of the invention, the invention may include an apparatus for the treatment where the magnets are permanent magnetic material with coercivity greater than 1 kOe.

In accordance with a further object of the invention, the apparatus can include a plurality of electromagnets.

In accordance with a further object of the invention, the apparatus can include at least one driven disk-like member with an outer peripheral surface where discrete stepwise changing permanent magnets form the magnetic material on the surface.

In accordance with a further object of the invention, the invention may include an apparatus for the treatment including at least one driven disk-like member with a groove on its outer peripheral surface, wherein a plurality of discrete stepwise changing electromagnets form the magnetic material.

In accordance with a further object of the invention, the invention may include an apparatus for the treatment including at least one driven disk-like member with an outer peripheral surface, where magnetic material of discrete stepwise changing permanent magnets is on the surface of the groove.

In accordance with a further object of the invention, the invention may include an apparatus for the treatment including at least one driven disk-like member with a groove on its outer peripheral surface, where a plurality of electromagnets form the magnetic material of discrete stepwise changing magnets on the surface of the groove.

In accordance with a further object of the invention, the invention may include an apparatus for treatment including a series of sequentially wound electromagnets fitted closely together and sequentially energized so as to create a uniformly stepwise increasing induced magnetic field with a maximum coercivity greater than 1 kOe and a frequency no less than 30 Hz.

In accordance with a further object of the invention, the invention may include an apparatus for treatment including a sequential array of electromagnets. A magnetic control device is disposed to sequentially apply a pulse of electric current to each electromagnet in the array in order to generate a moving magnetic field that changes in a stepwise fashion with each shift of the field along the array.

In accordance with a further object of the invention, the invention may include an apparatus for treatment including a toroidal sequential array of electromagnets. A magnetic control device disposed to sequentially apply a pulse of electric current to each electromagnet in the array in order to generate a moving magnetic field that changes in a stepwise fashion with each shift of the field along the array.

It is to be understood that both the foregoing general description and the following detailed description are exemplary only and are not restrictive of the invention, as claimed.

The accompanying drawings, which are incorporated in and constitute a part of this specification, illustrate several embodiments of the invention and together with the description serve to explain the principles of the invention.

### BRIEF DESCRIPTION OF THE DRAWINGS

FIG. 1 is a perspective view of a rotating disk like member;

FIG. 2 is a perspective view of the individual magnets of the disk like member shown in FIG. 1;

FIG. 3 is a plan view of a motor driven treatment system using the rotating disk like member;

FIG. 4 is a perspective view of an alternate embodiment of the rotating disk like member utilizing a plurality of permanent magnets forming a portion of the outer peripheral surface;

FIG. 5 is a cross-sectional view of the disk like member of FIG. 4 taken along line I-I;

FIG. 6 is a perspective view of an alternate embodiment with an electromagnet generates a stepwise changing magnetic field;

FIG. 7 is a graph plotting the magnetic and resultant electric fields generated by all the embodiments of this invention versus time;

FIG. 8 is a plan view of an embodiment of a treatment system using the stepwise changing electromagnet wherein full articulation of system is provided;

FIG. 9 is a perspective view of a transdermal medicant delivery device using a stepwise changing magnetic field to induce the transport of the medicant; and

FIG. 10 is a cross-sectional view of FIG. 9 taken along line J-J.

### DESCRIPTION OF THE PREFERRED EMBODIMENTS

Reference will now be made in detail to the present preferred embodiments of the invention, examples of which are illustrated in the accompanying drawings. Wherever possible, the same reference numbers will be used throughout the drawings to refer to the same parts.

The present invention creates an induced DC-like electric field in biological material to treat the material. The biological material can be portions of a living human or animal, such as body fluids, cells, tissue, or bone.

The induced DC-like electric field can treat the biological material in numerous ways, including promoting regeneration of damaged tissue. For example, the DC-like electric field can treat trauma (e.g., bruises, torn muscles, and cartilage damage); debilitation; organs by stimulating their regeneration to restore their functions; damaged or severed human nerves or axons; slow or non healing bone fractures (nonunions); occlusion of blood flow due to formation of plaque or other forms of calcification in the blood stream; ailments such as heart disease and senility, resulting from reduced blood flow to the affected organ; or osteoporosis (both prevention and reversal).

The induced DC-like electric field also can treat the biological material by destroying it or disrupting its normal processes. For example, cancerous tissues within the human body can be treated by inducing high electric currents.

The induced DC-like electric field also can be used to increase migration of electrically charged materials through the biological material. For example, the induced DC-like electric field can enhance transdermal transport of efficacious ionic drug components to specific locations within the tissue, thus reducing the amount of drug needed as well as toxic effects from the drug.

The induced DC-like electric field also can decrease human nerve pain by blocking electrical signals along nerve paths.

The present invention induces a DC-like electric field in the biological material by subjecting it to a stepwise time changing magnetic field. Ions exposed to a time changing magnetic field are subject to a force that will produce electric currents that will oppose the change in the magnetic field. The general law for the electric field associated with a changing magnetic field is the vector equation: ▾×E=−·B/·t, where E is the vector electric field and B the vector magnetic field.

The semi-conducting biological tissue will allow the flow of electric current within the tissue. If the induced EMF (Electro Motive Force) is not symmetric, the induced currents will produce a transport of electric charge that has been shown to be a necessary requirement for therapeutic application of electric fields. It is well known by medical researchers and medical practitioners that DC-like electric currents of strengths between 0.000001 amps and 0.001 amps can be of significant benefit in causing trauma healing to bone, nerve, and other tissue.

The present invention contemplates two different methods of generating the stepwise changing magnetic field to induce a DC-like electric field: (1) moving stepwise changing permanent magnets or electromagnets through space; and (2) a sequentially energized electromagnet(s) that produce a stepwise changing magnetic field.

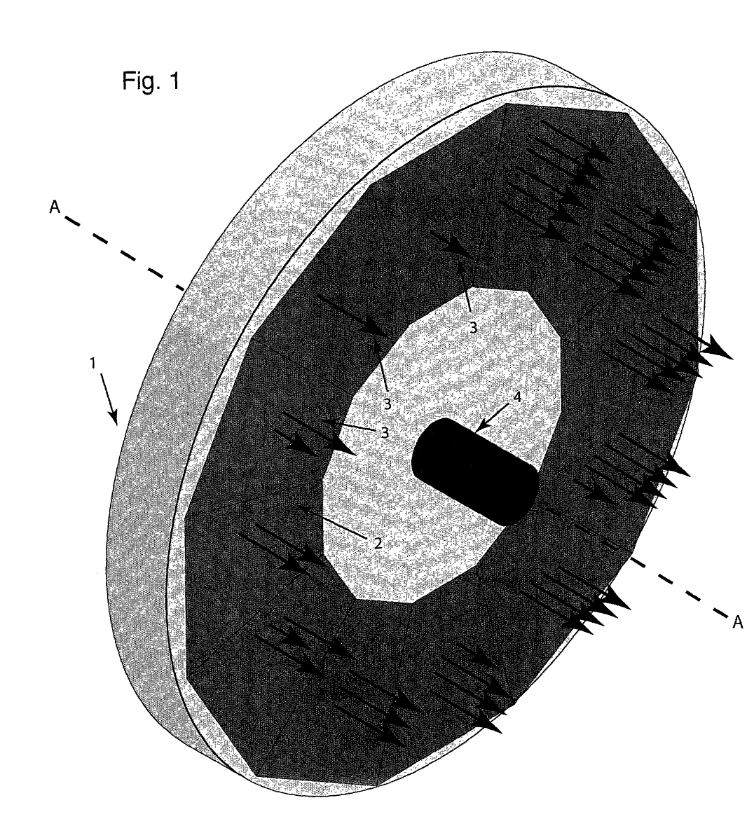

Embodiments of the invention using the first method of producing a stepwise changing magnetic field are depicted in FIGS. 1-5. In accordance with this aspect of the invention, the present invention includes at least two permanent or electromagnets for creating a stepwise changing magnetic field that can pass through the biological material, and a drive mechanism for moving the magnets relative to the material to induce a DC-like electric field within the material with the stepwise changing magnetic field.

As embodied herein, the permanent magnets for creating the stepwise changing magnetic field are rare earth magnets 2, having a coercivity of greater than 1 kOe. Preferably, the magnets 2 are neodymium-iron-boron (Ne₂ Fe₁₄ B). However, other permanent magnets of lesser strength can be used.

The embodiments of the invention disclosed and explained herein that practice the first method of moving a stepwise changing magnetic field use permanent magnets. However, the invention can also be carried out by replacing the permanent magnets with stepwise changing electromagnets. Any conventional electromagnets can be used that have the required strength. The electromagnets should be connected, by conventional connections, to a power source.

As embodied herein, the stepwise changing magnets 2, are mounted in a circular pattern in slots on the face of disk-like member 1. The stepwise changing magnets 2, are set into disk-like member 1 near the outer periphery of disk 1 so that all the outer surfaces of magnets 2 are aligned with and parallel to a single circular face of disk-like member 1. Each magnet 2 is connected, preferably with a suitable adhesive, in closely machined indentations on the circular face of the cylinder 1 so that they form a flat surface. Each magnet 2 preferably is glued into each slot with high quality glue. However, other conventional connectors can be used.

To achieve DC-like current, or “square wave” voltage each magnet 2 is arranged in a stepwise manner so that, looking at the surface of the magnets and proceeding in a counter-clockwise direction, each adjacent magnet has a constant magnetic field strength represented by arrows 3. The smallest arrow 3a indicates a vector magnetic field of ΔB. While Thus, the magnetic field strength of the first and weakest magnet is ΔB, the next 2ΔB, the next 3ΔB, and so on. The magnets are all polarized with the same polarity parallel to axis, A-A of the disk-like member 1. Disk-like member 1 is mounted on a shaft 4 parallel to axis A-A that can be driven.

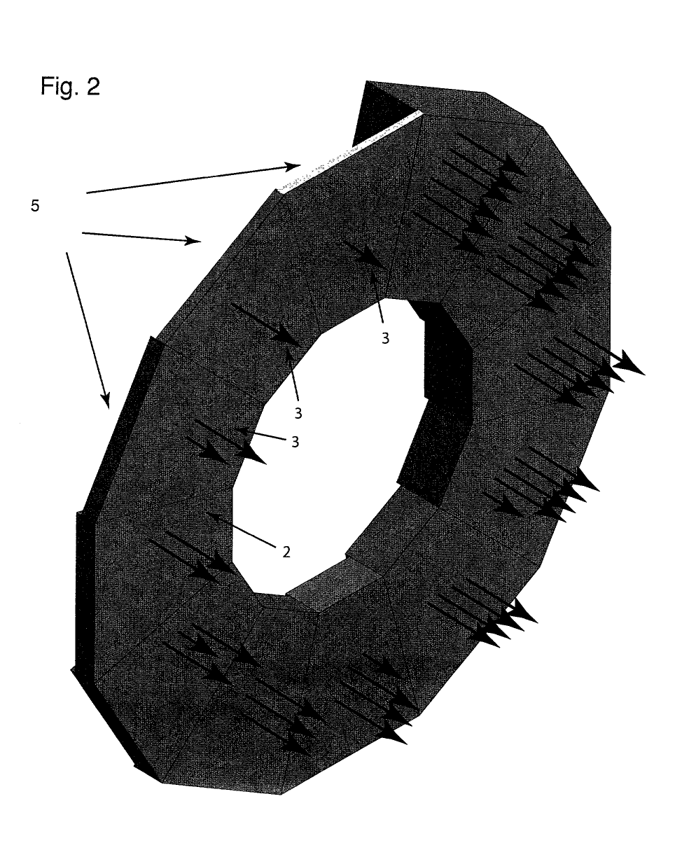

In the preferred embodiment, the permanent magnets 2 have varying depth or thickness 5 as shown in FIG. 2. Each magnet 2 is fitted into closely machined slots in disk-like member 1, which allow for the varying depth or thickness 5.

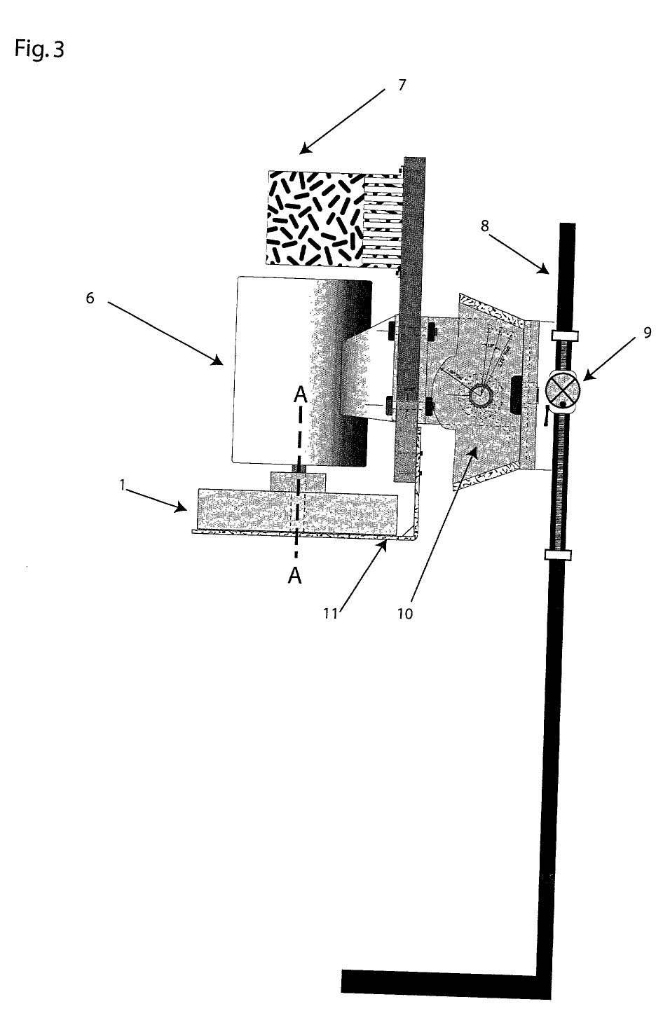

In the embodiment shown in FIG. 3, the disk-like member 3 is driven by a conventional electric or mechanical drive motor 6 connected to a speed control device 7. The speed control device 7 is adjustable through a wide range of rotary speeds and thereby can adjust the induced current strength. The rotation of the motor 6 is translated to the disk-like member 1 by the shaft 4. The angle of the outer magnetic face of the disk-like member is controlled by a dual axis assembly 10. The outer magnetic face of the disk-like member 1 is separated from the treated biological material by a protective guard 11. The protective guard 11 made of a non-conducting material such as glass-reinforced plastic or some other non-magnetic and non-conducting plastic. A gear track 9 mounted on a stand 8 controls the height of the outer magnetic face of disk-like member 1.

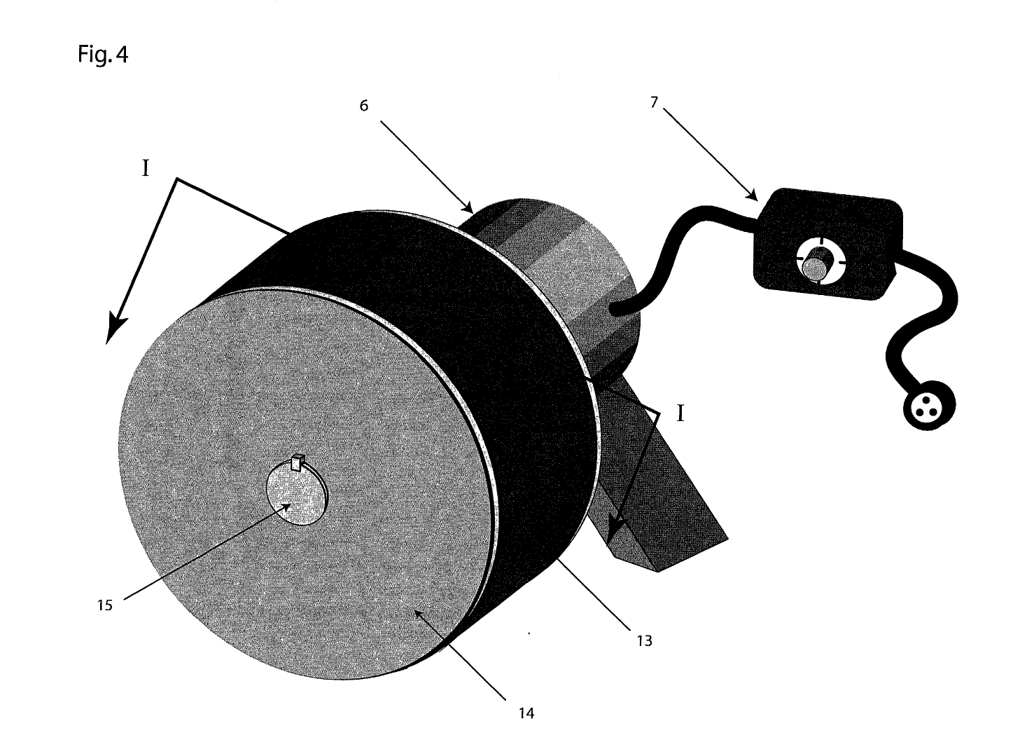

In another embodiment shown in FIG. 4, permanent magnets 13 are mounted on the rim or outer peripheral surface of the disk-like member 14. The disk-like member 14 is mounted on a shaft 15 and driven by motor 6 with a rotary speed controlled by speed controller 7.

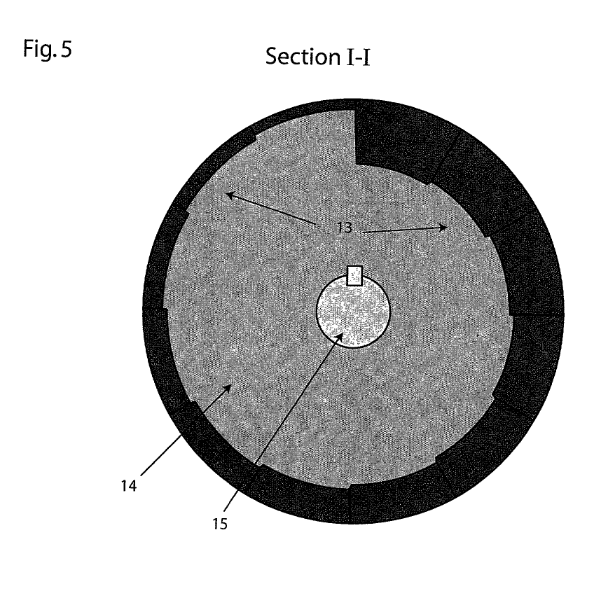

FIG. 5 is a sectional view taken along line I-I of FIG. 4. FIG. 5 shows a section of the disk-like member 14. In the embodiment, the stepwise change in magnetic field strength is created by the changing radial thickness of each magnetic segment 13. Each magnetic segment 13 has a polarity of a north pole facing radially outward from the center of in disk-like member 14. Preferably, the magnets 13 are glued into closely machined slots in disk-like member 14 although other attachments may be used.

The rotation of disk-like member 14 produces a stepwise changing magnetic field near the outer rim surface. The rate of rotation will determine the time rate of change of the magnetic field and thus the strength of the induced electric field is proportional to the R.P.M. (Revolutions Per Minute) of the disk-like member 14.

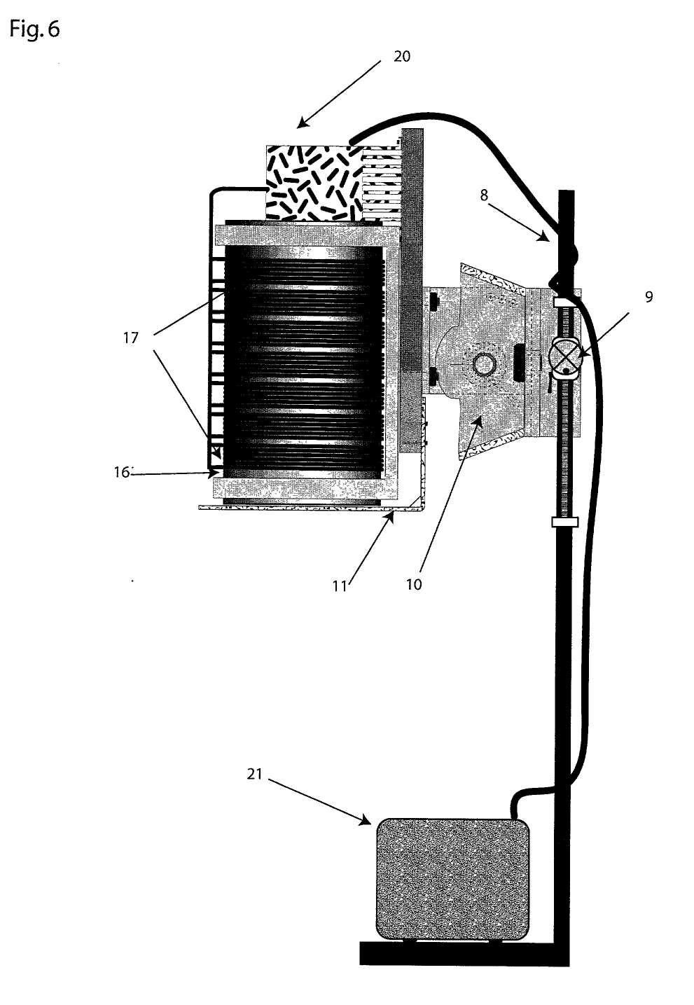

FIG. 6 shows a further preferred embodiment. This preferred embodiment produces a stepwise changing magnetic field with electromagnet coils 17 mounted on a core 16. In this embodiment, the plurality of electromagnet coils 17 are progressively energized by switches 19 in a time sequence by controller 20. The progressive increase in the number of amp-turns of coils 17 that are energized by the application of the electric power through timer and switches 19 produces a steady increase in the magnetic field 18.

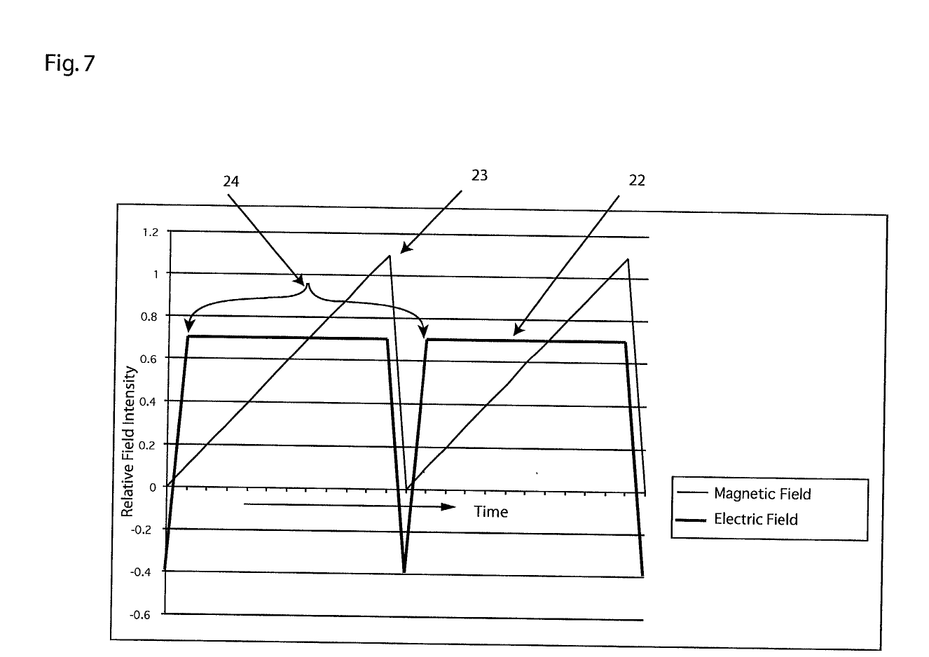

FIG. 7 is a graph plotting the magnetic field 23 and the resulting electric field 22 plotted versus time through two cycles 24. Any semi-conducting biological material that is suffused by the magnetic field 18 will support electric currents that are driven by an induced electric field 22. The resulting induced DC-like electric field 22 will produce charge transport in the semi-conducting medium. The frequency of this electric field as indicated by time of one cycle 24 is not less than 50 Hz and not greater than 1000 Hz.

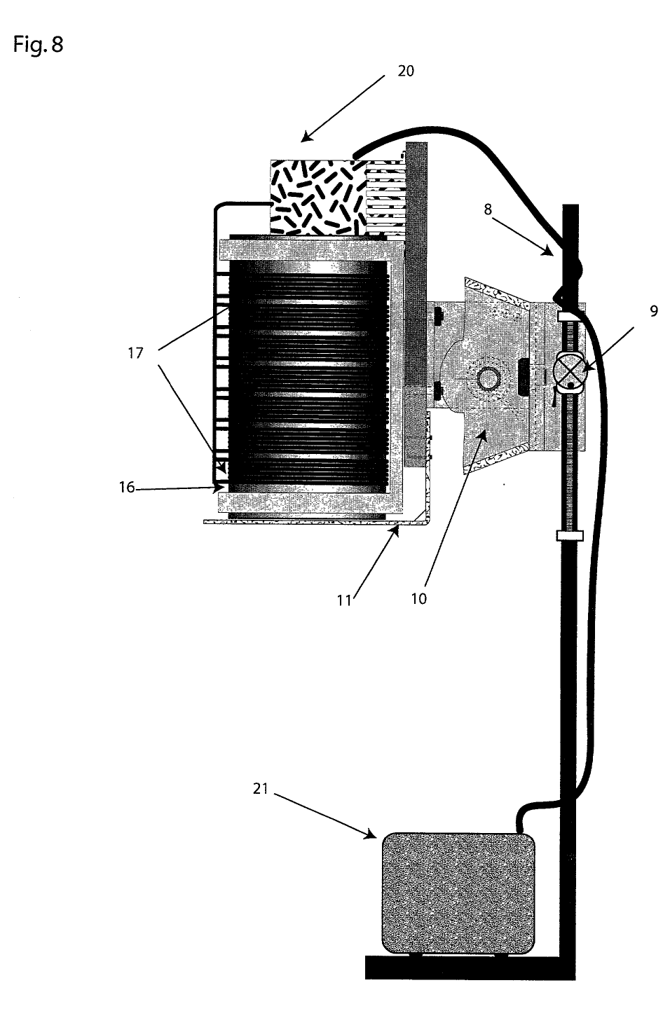

FIG. 8 shows a typical embodiment for the therapeutic application. Power supply 21 energizes coils 17 on core 16 through controller 20. The angle of the outer magnetic face of the core 16 is controlled by a dual axis assembly 10. The outer magnetic face core 16 is separated from the treated biological material by a protective guard 11. The protective guard is made of non-conducting material such as glass-reinforced plastic or some other non-magnetic and non-conducting plastic. The height of the outer magnetic face of core 16 is controlled with gear track 9 mounted on stand 8.

Other embodiments, not shown, would allow a single coil 17 to produce a DC-like electric field 22 if the singular coil 17 is energized by a continuously increasing electric field that would produce a continuously increasing magnetic field such as shown in FIG. 7.

Another embodiment, not shown, shapes the magnetic core 16 or includes magnetic shielding materials to focus or confinement of the magnetic field 18. The judicious use of such well known technologies by one skilled in the art would allow the increase of magnetic filed intensity in the treatment volume of the biological material.

In a further embodiment, the apparatus includes a transporter for moving at least one medicant to humans and animals through a transdermal site. The apparatus includes a medicant supply located on the site and at least one stepwise changing set of permanent magnets in proximity to the site. A drive mechanism is disposed to move the magnets relative to the site to induce a DC-like electric field with in the site, the electric field being of sufficient magnitude to increase the rate of transportation of the medicant.

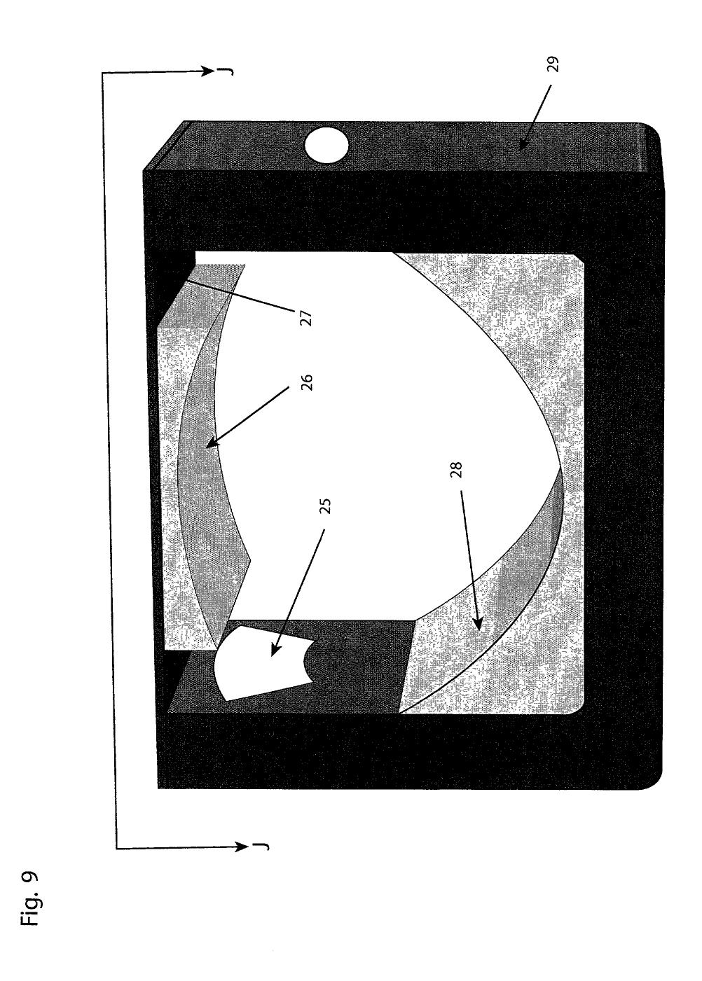

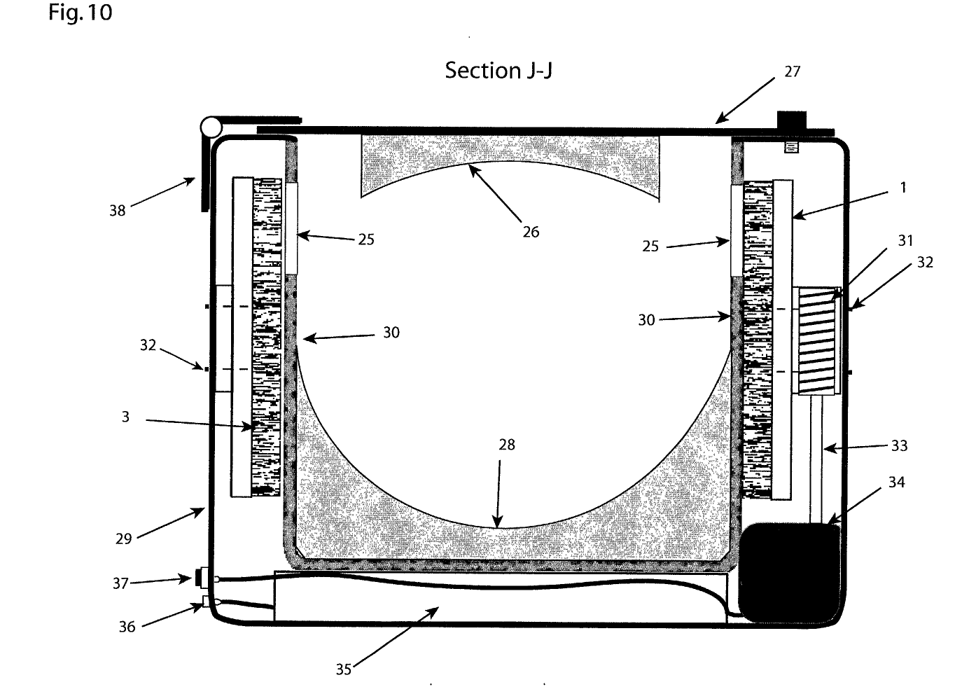

As embodied herein, the medicant supply is a drug-saturated pad 26 that can be held in place against the surface of the biological material. As shown in FIGS. 9 and 10, the portable transdermal drug induction system 29 includes a plate 27 for holding the drug-saturated pad 26. The plate 27 rotates on hinge 38 to allow the placement or removal of the pad 26.

As embodied herein, the permanent magnets 3 mounted on disk 1 rotate on bearings 32. The permanent magnets 3 are mounted so that magnets 3 of equal strength but opposite polarity face each other across the encased volume. Thus the magnetic field exiting magnetically transparent windows 25 is in a single direction at all times and is attractive at all times. One of the disks is driven by drive mechanism 32 mounted on a shaft 33 driven by electric motor 34. A second drive mechanism 32 may be used on the opposite magnetic disk. The speed of the electric motor 34 is varied by a controller 37 and powered by batteries 35, which may be recharged via a connection 36.

The limb or body portion is placed between drug pad 26 and a cushion 28 so that the skin surface is located in the plane between magnetically transparent windows 25 located within the magnetic shielding material 30. The stepwise time changing magnetic filed exiting from magnetically transparent windows 25 and emanating form the opposite magnetic pole surfaces of the disk-like member 1 transects both the drug saturated pad 26 and the dermal regions of the encased biological material. The magnetically generated induced DC-like electric field causes ionic forms of the drug held in the pad 26 to penetrate the skin and tissue of the limb or body portion enclosed between the drug pad 26 and the cushion 28.

One or both disks 1 are driven by the electrical motor 34. The controller 37 with a variable speed adjustment allows a wide assortment of rotary speeds, direction of rotation, and times of operation.

In the preferred embodiment, the rotation or the driven disk 1 causes the facing magnet of opposite polarity freewheeling on bearing shaft 32 to rotate.

A housing 29 encloses the unit. The inner surfaces of the housing 29 that are most proximate and parallel to the outermost faces of the magnetic disks are covered by the magnetic shielding 30, which prevents the passage of the magnetic field except through such openings as are provided. The magnetic shielding preferably has two annular opening that allow the magnetic field to exit the housing surfaces and cause transdermal transport of the cations or anions of various drugs through the surface of the skin of the enclosed limb.

In a further embodiment of the present invention, an apparatus transporting at least one medicant to humans and animals through a transdermal site. The apparatus includes a medicant supply located on the site and electromagnets in proximity to the site. A control device is disposed to apply stepwise increasing current to the electromagnets to generate a stepwise changing magnetic field through the transdermal site thereby inducing a DC-like electric field within the material in proximity to the site, the electric field being of sufficient magnitude to increase the rate of transportation of the medicant. This embodiment of the invention is similar to that shown in FIGS. 9 and 10 but employs electromagnets of the type shown in FIG. 6 instead of permanent magnets.

Each of the above embodiments and numerous other possible configurations are based on the concept of a stepwise changing magnetic field generating an electric current in or on a biological material without the use of electrodes. Stepwise changing magnetic fields of up to 2000 gauss can be achieved by both permanent and electromagnets. Rates of change for fields generated by both permanent and electromagnets can be achieved using standard methods of movement or electronic switching or electric current modulation. The systems can meet or exceed all existing invasive therapeutic devices using direct electric stimulation. The systems preferably generate an electric field strength in the biological material in the range of 0.001 V/m to 100.0 V/m (volts per meter). The systems can also preferably generate a DC-like electric current in the biological material in the range of 0.000001 amperes to 10.0 amperes. Thus, the present invention generates DC-like electric currents for medical treatment while elimination the risk to the patient caused by inserting electrodes.

It will be apparent to those skilled in the art that various modifications and variations can be made in the present invention without departing from the scope or spirit or the invention. Other embodiments of the invention will be apparent to those skilled in the art from consideration of the specifications and practice of the invention disclosed herein. It is intended that the specification and examples be considered as exemplary only, with a true scope and spirit of the invention being indicated by the following claims.

I claim:

1. An apparatus for treating biological material, comprising:
two magnets each producing a magnetic field combining to form a magnetic field on the biological material; and
a drive for changing the magnetic field on the biological material.

2. The apparatus according to claim 1, wherein said magnets are permanent magnets.

3. The apparatus according to claim 1, wherein said magnets are electromagnets.

4. The apparatus according to claim 1, wherein one of said magnets produces a stronger magnetic field and the other produces a weaker magnetic field.

5. The apparatus according to claim 4, wherein said drive includes a rotating disk-like member holding said magnets.

6. The apparatus according to claim 5, wherein said disc-like member has a periphery, and a plurality of said magnets each produce a different magnetic field and are disposed about said periphery in order of increasing magnetic field.

7. The apparatus according to claim 1, wherein said magnets are have a coercivity greater than 1 kOe.

8. The apparatus according to claim 5, wherein said disc-like member has a face with a groove formed therein about said periphery, said magnets being held in said groove.

9. The apparatus according to claim 3, wherein said drive is a controller sequentially energizing said electromagnets.

10. The apparatus according to claim 9, including three of said electromagnets, said electromagnets being energized by said controller to create a stepwise increasing magnetic field.

11. The apparatus according to claim 9, wherein said controller cycles said electromagnets.

12. The apparatus according to claim 9, wherein said electromagnets are coiled cylindrically in series around the biological tissue.

13. The apparatus according to claim 9, wherein said electromagnets are disposed circumferentially around the biological tissue.

14. The apparatus according to claim 11, wherein the electromagnets are cycled at a frequency no less than 30 Hz.

15. The apparatus according to 5, wherein said disc-like member is rotated to produce an electric field in the biological material having a frequency of no less than 30 Hz.

16. The apparatus according to claim 1, including a protective guard between said magnets and the biological tissue.

17. The apparatus according to claim 1, including a pad for temporarily storing an ion-containing medicant, the ion-containing medicant being moved by the magnetic field into the biological tissue.

18. The apparatus according to claim 17, including a magnetic shield between the biological tissue and said magnets and having a magnetically transparent window formed therein facing the biological tissue for polarizing the magnetic field passing therethrough.

19. A method for inducing a DC-like electric field in biological tissue, which comprises:
increasing a magnetic field on the biological tissue; and
suddenly decreasing the magnetic field.

20. The method according to claim 19, which further comprises increasing the magnetic field steadily.

21. The method according to claim 19, which further comprises cycling the increasing and the sudden decreasing.

22. The method according to claim 21, which further comprises cycling at a frequency of no less than 30 Hz.

23. The method according to claim 19, which further comprises forming the magnetic field with a magnet having a coercivity in the greater than 1 kOe.

24. The method according to claim 19, which further comprises placing ion-containing medicant on the biological tissue.

25. The method according to claim 24, which further comprises:
temporarily storing the medicant in a pad; and
placing the pad against the biological tissue.
